(19) United States
(12) Patent Application Publication
(10) Pub. No.: US 2025/0032393 A1
(43) Pub. Date: Jan. 30, 2025

(54) FREE STANDING FILM

(71) Applicants: Dow Silicones Corporation, Midland, MI (US); Dow Silicones Corporation, Midland, MI (US); Rohm And Haas Company, Collegeville, PA (US)

(72) Inventors: Saugata Nad, Brussels (BE); Serge Creutz, Liege (BE); Christel Simon, Lobbes (BE)

(21) Appl. No.: 18/836,049

(22) PCT Filed: Feb. 9, 2023
(86) PCT No.: PCT/US2023/012654

§ 371 Date: Aug. 6, 2024

Related U.S. Application Data
(63) Continuation of application No. PCT/US2023/012752, filed on Feb. 10, 2023.
(60) Provisional application No. 63/310,668, filed on Feb. 16, 2022.

(51) Int. Cl. A61K 8/81; A61K 8/04; A61K 8/44; A61K 8/73; A61Q 5/06

(52) U.S. Cl. CPC A61K 8/8147; A61K 8/042; A61K 8/44; A61K 8/732; A61Q 5/06; A61K 2800/262; A61K 2800/48

(57) ABSTRACT

A clear hair styling formulation is provided, comprising: a dermatologically acceptable vehicle; and a hair fixative polymer; wherein the hair fixative polymer is a hydrolyzed starch; wherein the hydrolyzed starch has a weight average molecular weight of 5,000 to 50,000 Daltons.

The present invention relates to a clear hair styling formulation. In particular, the present invention relates to a clear hair styling formulation, comprising: a dermatologically acceptable vehicle; and a hair fixative polymer; wherein the hair fixative polymer is a hydrolyzed starch; wherein the hydrolyzed starch has a weight average molecular weight of 5,000 to 50,000 Daltons.

Clear hair styling formulations are popular with consumers, as consumers perceive that clarity of the formulation represents purity and quality in hair care products. Yet clarity alone is not enough. Other properties such as curl strength or hold and resistance to humidity are very important to hair styling products.

Conventional hair styling formulations contain synthetic polymers for imparting a style to hair. The synthetic polymers are typically based on vinyl monomers such as polyvinylpyrrolidone (PVP) or n-vinylpyrrolidone (VP). While such polymers provide good hold while remaining water soluble enough to be easily washed out of the hair, they present a perception of having an unfavorable sustainability profile. Accordingly, it would be desirable to provide alternative polymers for imparting style to hair that exhibit the perception of having a more favorable sustainability profile. However, many natural polymers are incompatible with synthetic fixative polymers when blended into hair styling formulations resulting in hazy formulations and/or create non-uniform films on hair, which non-uniformity leads to decreased performance in humidity resistance.

In U.S. Pat. No. 7,972,589, Leighton et al disclose a hair fixative film with blend of natural and synthetic polymers. In particular, Leighton et al disclose a hair fixative film comprising: (a) at least one hair fixative polymer comprising a mixture of synthetic and natural polymers, wherein the ratio of synthetic to natural polymer is from about 20:80 to about 75:25 and wherein the synthetic polymers are selected from the group consisting of acrylates copolymer, polyurethane, polystyrene, polyvinyl amide, polyvinyl ester, acrylates/octylacrylamide copolymer and mixtures thereof; and (b) a plasticizer; wherein the hair fixative film is an unsupported film and has a thickness of about 1 to about 500 microns, wherein the hair fixative film provides hair stiffness of from about 0.004 Joules to about 0.030 Joules when applied to hair, wherein the hair fixative film dissolves when exposed to water, and wherein the hair fixative film has a high humidity curl retention of greater than 30 percent after 2 hours.

Notwithstanding, there is a continuing need for new clear hair styling formulations. In particular, for new clear hair styling formulation containing a compatible blend of natural hair fixative polymers with conventional synthetic polymers, which formulations are both clear and provide effective curl strength or hold and resistance to humidity.

The present invention provides a clear hair styling formulation, comprising: a dermatologically acceptable vehicle; a hair fixative polymer; wherein the hair fixative polymer is a hydrolyzed starch; wherein the hydrolyzed starch has a weight average molecular weight of 5,000 to 50,000 Daltons.

The present invention provides a clear hair styling formulation, comprising: a dermatologically acceptable vehicle; a hair fixative polymer; wherein the hair fixative polymer is a hydrolyzed starch; wherein the hydrolyzed starch has a weight average molecular weight of 5,000 to 50,000 Daltons; and a thickener.

The present invention provides a clear hair styling formulation, comprising: a dermatologically acceptable vehicle; a hair fixative polymer; wherein the hair fixative polymer is a hydrolyzed starch; wherein the hydrolyzed starch has a weight average molecular weight of 5,000 to 50,000 Daltons; and a thickener; wherein the thickener includes a crosslinked homopolymer polymerized from (meth)acrylic acid.

The present invention provides a method of styling hair, comprising: providing a clear hair styling formulation of the present invention; applying the clear hair styling formulation to hair; and imparting a style to the hair.

### DETAILED DESCRIPTION

We have surprisingly found that hair fixative polymer hydrolyzed starch having a weight average molecular weight of 5,000 to 50,000 Daltons (preferably, 7,500 to 35,000 Daltons; more preferably, 8,000 to 18,000 Daltons; most preferably, 9,000 to 15,000 Daltons) is exceptionally compatible with conventional thickeners (e.g., carbomer), such that hair styling formulations prepared with blends thereof with conventional thickeners provide excellent high humidity curl retention (i.e., of greater than 50% (preferably, greater than 75%; more preferably, greater than 80%; most preferably, >90%) after eight hours at 25° C. in a 90% relative humidity environment) and have a turbidity, when measured by nephelometry at a concentration of 1 wt % thickener, 3 wt % of the hair fixative polymer hydrolyzed starch and a pH of 7 to 7.5, of 0 to 20 Nephelometric Turbidity Units (NTU).

Unless otherwise indicated, ratios, percentages, parts, and the like are by weight.

As used herein, unless otherwise indicated, the phrase “molecular weight” or M_{W} refers to the weight average molecular weight as measured in a conventional manner with gel permeation chromatography (GPC) and conventional standards, such as pullulan narrow molecular weight standards with molecular weight range of 180 to 708,000 Daltons. GPC techniques are discussed in detail in Modern Size-Exclusion Liquid Chromatography-Practice of Gel Permeation and Gel Filtration Chromatography, Second Edition, A. M. Striegel, W. W. Yau, J. J. Kirkland, D. D. Bly; John Wiley & Sons, Inc. 2009. Molecular weights are reported herein in units of Daltons, or equivalently, g/mol.

The term “dermatologically acceptable” as used herein and in the appended claims refers to ingredients that are typically used for topical application to the skin, and is intended to underscore that materials that are toxic when present in the amounts typically found in skin care compositions are not contemplated as part of the present invention.

Preferably, the clear hair styling formulation of the present invention is selected from the group consisting of a hair spray, a hair fixative, a hair mousse, a hair gel/cream/paste, a hair volumizing composition, a hair leave-on cream, a hair mask, a hair lotion, a hair serum, a hair oil, a hair clay, a hair pomade, a hair wax, a hair powder and a dry shampoo. Preferably, the clear hair styling formulation of the present invention is formulated to facilitate styling hair to hold or fix the hair in a particular shape.

Preferably, the clear hair styling formulation of the present invention has a turbidity, when measured by nephelometry at a concentration of 1 wt % hair fixative polymer and a pH of 7 to 7.5, of 0 to 20 (preferably, 0 to 15; more preferably, 0 to 10; most preferably, 0 to 7.5) Nephelometric Turbidity Units (NTU); wherein the turbidity is measured according to the process described in the Examples.

Preferably, the clear hair styling formulation of the present invention, comprises: a dermatologically acceptable vehicle (preferably. 25 to 99.5 wt % (more preferably, 48 to 98.15 wt %; still more preferably, 79 to 95.9 wt %; most preferably, 84.5 to 93 wt %), based on weight of the clear hair styling formulation, of the dermatologically acceptable vehicle); a hair fixative polymer (preferably, 0.5 to 10 wt % (more preferably, 1 to 8 wt %; still more preferably, 2 to 7.5 wt %; most preferably, 3 to 6 wt %), based on weight of the clear hair styling formulation, of the hair fixative polymer); wherein the hair fixative polymer is a hydrolyzed starch (preferably, wherein the hydrolyzed starch is selected from the group consisting of hydrolyzed corn starch, hydrolyzed potato starch, hydrolyzed rice starch, hydrolyzed quinoa starch, hydrolyzed pea starch and mixtures thereof; more preferably, wherein the hydrolyzed starch is selected from the group consisting of hydrolyzed corn starch, hydrolyzed potato starch and mixtures thereof; most preferably, hydrolyzed corn starch); optionally, a thickener (preferably, 0 to 30 wt % (more preferably, 0.2 to 25 wt %; still more preferably, 0.5 to 15 wt %; most preferably, 0.75 to 2 wt %), based on weight of the clear hair styling formulation, of the thickener); optionally, a plasticizer (preferably, 0 to 30 wt % (more preferably, 0.5 to 20 wt %; still more preferably, 1 to 10 wt %; most preferably, 2 to 5 wt %), based on weight of the clear hair styling formulation, of the plasticizer); optionally, an antimicrobial agent/preservative (preferably, 0 to 5 wt % (more preferably, 0.05 to 4 wt %; still more preferably, 0.1 to 3 wt %; most preferably, 0.5 to 2 wt %), based on weight of the clear hair styling formulation, of the antimicrobial agent/preservative); optionally, a pH adjusting agent (preferably, 0 to 5 wt % (more preferably, 0.1 to 4 wt %; still more preferably, 0.5 to 3 wt %; most preferably, 0.75 to 2 wt %), based on weight of the clear hair styling formulation, of the antimicrobial agent/preservative); wherein the hydrolyzed starch has a weight average molecular weight of 5,000 to 50,000 Daltons (preferably, 7,500 to 35,000 Daltons; more preferably, 8,000 to 18,000 Daltons; most preferably, 9,000 to 15,000 Daltons) (preferably, measured by GPC using pullulan narrow molecular weight standards with molecular weight range of 180 to 708,000 Daltons).

Preferably, the clear hair styling formulation of the present invention, comprises: 25 to 99.5 wt % (more preferably, 48 to 98.15 wt %; still more preferably, 79 to 95.9 wt %; most preferably, 84.5 to 93 wt %), based on weight of the clear hair styling formulation, of a dermatologically acceptable vehicle. More preferably, the clear hair styling formulation of the present invention, comprises: 25 to 99.5 wt % (more preferably, 48 to 98.15 wt %; still more preferably, 79 to 95.9 wt %; most preferably, 84.5 to 93 wt %), based on weight of the clear hair styling formulation, of a dermatologically acceptable vehicle; wherein the dermatologically acceptable vehicle comprises water. Still more preferably, the clear hair styling formulation of the present invention, comprises: 25 to 99.5 wt % (more preferably, 48 to 98.15 wt %; still more preferably, 79 to 95.9 wt %; most preferably, 84.5 to 93 wt %), based on weight of the clear hair styling formulation, of a dermatologically acceptable vehicle; wherein the dermatologically acceptable vehicle is selected from the group consisting of water and an aqueous C₁₋₄ alcohol mixture. Most preferably, the clear hair styling formulation of the present invention, comprises: 25 to 99.5 wt % (more preferably, 48 to 98.15 wt %; still more preferably, 79 to 95.9 wt %; most preferably, 84.5 to 93 wt %), based on weight of the clear hair styling formulation, of a dermatologically acceptable vehicle; wherein the dermatologically acceptable vehicle is water.

When the clear hair styling formulation is a sprayable formulation, the dermatologically acceptable vehicle preferably comprises 80 to 100 wt %, based on weight of the dermatologically acceptable vehicle, of water and 0 to 20 wt %, based on weight of the dermatologically acceptable vehicle, of a C₁₋₄ alcohol. More preferably, when the clear hair styling formulation is a sprayable formulation, the dermatologically acceptable vehicle preferably comprises 80 to 100 wt %, based on weight of the dermatologically acceptable vehicle, of water and 0 to 20 wt %, based on weight of the dermatologically acceptable vehicle, of ethanol.

Preferably, the water used in the clear hair styling formulation of the present invention is at least one of distilled water and deionized water. More preferably, the water used in the clear hair styling formulation of the present invention is distilled and deionized.

Preferably, the clear hair styling formulation of the present invention comprises 0.5 to 10 wt % (preferably, 1 to 8 wt %; more preferably, 2 to 7.5 wt %; preferably, 3 to 6 wt %), based on weight of the clear hair styling formulation, of the hair fixative polymer; wherein the hair fixative polymer is a hydrolyzed starch; wherein the hydrolyzed starch has a weight average molecular weight of 5,000 to 50,000 Daltons (preferably, 7,500 to 35,000 Daltons; more preferably, 8,000 to 18,000 Daltons; most preferably, 9,000 to 15,000 Daltons) (preferably, wherein the weight average molecular weight is measured by GPC using pullulan narrow molecular weight standards with molecular weight range of 180 to 708,000 Daltons). More preferably, the clear hair styling formulation of the present invention comprises 0.5 to 10 wt % (preferably, 1 to 8 wt %; more preferably, 2 to 7.5 wt %; preferably, 3 to 6 wt %), based on weight of the clear hair styling formulation, of the hair fixative polymer; wherein the hair fixative polymer is a hydrolyzed starch selected from the group consisting of a hydrolyzed corn starch, a hydrolyzed potato starch, a hydrolyzed rice starch, a hydrolyzed quinoa starch and mixtures thereof; wherein the hydrolyzed starch has a weight average molecular weight of 5,000 to 50,000 Daltons (preferably, 7,500 to 35,000 Daltons; more preferably, 8,000 to 18,000 Daltons; most preferably, 9,000 to 15,000 Daltons) (preferably, wherein the weight average molecular weight is measured by GPC using pullulan narrow molecular weight standards with molecular weight range of 180 to 708,000 Daltons). Still more preferably, the clear hair styling formulation of the present invention comprises 0.5 to 10 wt % (preferably, 1 to 8 wt %; more preferably, 2 to 7.5 wt %; preferably, 3 to 6 wt %), based on weight of the clear hair styling formulation, of the hair fixative polymer; wherein the hair fixative polymer is a hydrolyzed starch selected from the group consisting of a hydrolyzed corn starch, a hydrolyzed potato starch and mixtures thereof; wherein the hydrolyzed starch has a weight average molecular weight of 5,000 to 50,000 Daltons (preferably, 7,500 to 35,000 Daltons; more preferably, 8,000 to 18,000 Daltons; most preferably, 9,000 to 15,000 Daltons) (preferably, wherein the weight average molecular weight is measured by GPC using pullulan narrow molecular weight standards with molecular weight range of 180 to 708,000 Daltons). Most preferably, the clear hair styling formulation of the present invention comprises 0.5 to 10 wt % (preferably, 1 to 8 wt %; more preferably, 2 to 7.5 wt %; preferably, 3 to 6 wt %), based on weight of the clear hair styling formulation, of the hair fixative polymer; wherein the hair fixative polymer is a hydrolyzed corn starch; wherein the hydrolyzed starch has a weight average molecular weight of 5,000 to 50,000 Daltons (preferably, 7,500 to 35,000 Daltons; more preferably, 8,000 to 18,000 Daltons; most preferably, 9,000 to 15,000 Daltons) (preferably, wherein the weight average molecular weight is measured by GPC using pullulan narrow molecular weight standards with molecular weight range of 180 to 708,000 Daltons).

Preferably, the hydrolyzed starch contains 0 to 100 wt % (preferably, 20 to 100 wt %; more preferably, 40 to 95 wt %; still more preferably, 50 to 90 wt %; most preferably, 60 to 75 wt %), based on weight of the hydrolyzed starch, of amylopectin.

Preferably, the hydrolyzed starch has a polydispersity, M_{w}/Mₙ, of >2 to 10 (preferably, 2.25 to 10; more preferably, 2.5 to 9; most preferably, 2.75 to 8).

Preferably, the clear hair styling formulation of the present invention further comprises 0 to 30 wt % (preferably, 0.2 to 25 wt %; more preferably, 0.5 to 15 wt %; most preferably, 0.75 to 2 wt %), based on weight of the clear hair styling formulation, of a thickener. More preferably, the clear hair styling formulation of the present invention further comprises 0 to 30 wt % (preferably, 0.2 to 25 wt %; more preferably, 0.5 to 15 wt %; most preferably, 0.75 to 2 wt %), based on weight of the clear hair styling formulation, of a thickener; wherein the thickener is selected to increase the viscosity of the clear hair styling formulation, preferably without substantially modifying the other properties of the clear hair styling formulation. Still more preferably, the clear hair styling formulation of the present invention further comprises 0 to 30 wt % (preferably, 0.2 to 25 wt %; more preferably, 0.5 to 15 wt %; most preferably, 0.75 to 2 wt %), based on weight of the clear hair styling formulation, of a thickener; wherein the thickener is selected from the group consisting of synthetic polymers (e.g., polyethylene glycol, water soluble acrylic polymers, polyurethane, polystyrenic, water soluble polyesters, polyamides, polyacrylamides, polyamines, poly(quaternary amines), styrene maleic anhydride resin, polyethylene amines, carbomers, poloxamers, polyethyleneimines (e.g., PEI-10)); natural gums (e.g., tragacanth, carrageenan, cetyl alcohol, xanthan gum, petroleum jelly, waxes, bentonite, colloidal silicon dioxide); polysaccharides (e.g., cellulose, guar); salts (e.g., sodium chloride); and combinations thereof. Yet more preferably, the clear hair styling formulation of the present invention further comprises 0 to 30 wt % (preferably, 0.2 to 25 wt %; more preferably, 0.5 to 15 wt %; most preferably, 0.75 to 2 wt %), based on weight of the clear hair styling formulation, of a thickener; wherein the thickener includes at least one of a crosslinked poly((meth)acrylic acid) polymer and a hydrophobically modified crosslinked poly(meth)acrylic acid) polymer. Yet still more preferably, the clear hair styling formulation of the present invention further comprises 0 to 30 wt % (preferably, 0.2 to 25 wt %; more preferably, 0.5 to 15 wt %; most preferably, 0.75 to 2 wt %), based on weight of the clear hair styling formulation, of a thickener; wherein the thickener includes a synthetic polymer selected from the group consisting of (i) a crosslinked homopolymer polymerized from (meth)acrylic acid (preferably, a homopolymer of (meth)acrylic acid crosslinked with allyl ether or pentaerythritol, an allyl ether of sucrose, or an allyl ether of propylene); (ii) a crosslinked copolymer polymerized from a first monomer selected from one or more monomers of (meth)acrylic acid, substituted (meth)acrylic acid, salts of (meth)acrylic acid and salts of substituted acrylic acid and a second monomer selected from one or more C₁₀₋₃₀ alkyl esters of (meth)acrylic acid; and mixtures thereof. Most preferably, the clear hair styling formulation of the present invention further comprises 0 to 30 wt % (preferably, 0.2 to 25 wt %; more preferably, 0.5 to 15 wt %; most preferably, 0.75 to 2 wt %), based on weight of the clear hair styling formulation, of a thickener; wherein the thickener includes a crosslinked homopolymer polymerized from (meth)acrylic acid (preferably, a homopolymer of (meth)acrylic acid crosslinked with allyl ether or pentaerythritol, an allyl ether of sucrose, or an allyl ether of propylene).

Preferably, the clear hair styling formulation of the present invention further comprises a thickener, wherein the weight ratio of the thickener to the hair fixative polymer is 5:95 to 95:5 (preferably, 1:1 to 1:50; more preferably, 1:5 to 1:25; most preferably, 1:5 to 1:10).

Preferably, the clear hair styling formulation of the present invention further comprises 0 to 30 wt % (preferably, 0.5 to 20 wt %; more preferably, 1 to 10 wt %; most preferably, 2 to 5 wt %), based on weight of the clear hair styling formulation, of a plasticizer. More preferably, the clear hair styling formulation of the present invention further comprises 0 to 30 wt % (preferably, 0.5 to 20 wt %; more preferably, 1 to 10 wt %; most preferably, 2 to 5 wt %), based on weight of the clear hair styling formulation, of a plasticizer; wherein the plasticizer is selected from the group consisting of dimethicone copolyols (e.g., PEG-12 dimethicone, PEG/PPG-18/18 dimethicone, PPG-12 Dimethicone); polyols (e.g., ethylene glycol, propylene glycol, glycerin, dipropylene glycol, sorbitol, mannitol, maltitol, lactitol, fructose, glucose, sucrose, maltose, lactose, high fructose corn syrup solids, ascorbic acid); polycarboxylic acids (e.g., citric acid, maleic acid, succinic acid, polyacrylic acid, polymaleic acid); polyesters (e.g., glycerol triacetate, acetylated-monoglyceride, diethyl phathalate, triethyl citrate, tributyl citrate, acetyl triethyl citrate, acetyl tributyl citrate) and mixtures thereof). Most preferably, the clear hair styling formulation of the present invention optionally comprises 0 to 30 wt % (preferably, 0.5 to 20 wt %; more preferably, 1 to 10 wt %; most preferably, 2 to 5 wt %), based on weight of the clear hair styling formulation, of a plasticizer; wherein the plasticizer includes glycerin.

Preferably, the clear hair styling formulation of the present invention, optionally, further comprises at least one additional ingredient selected from the group consisting of an antimicrobial agent/preservative (e.g., benzoic acid, sorbic acid, phenoxyethanol, methylisothiazolinone, ethylhexyl glycerin); a colorant; a pH adjusting agent; an antioxidant (e.g., butylated hydroxytoluene); a humectant (e.g., glycerin, sorbitol, monoglycerides, lecithins, glycolipids, fatty alcohols, fatty acids, polysaccharides, sorbitan esters, polysorbates (such as Polysorbate 20, Polysorbate 40, Polysorbate 60, and Polysorbate 80), diols (such as propylene glycol), diol analogs, triols, triol analogs, cationic polymeric polyols)); a wax; a foaming agent; an emulsifying agent; a fragrance; a chelating agent (e.g., tetrasodium ethylene diamine tetraacetic acid); a bleaching agent; a lubricating agent; a sensory modifier (e.g., silicone); a vitamin; a bioactive agent; an anti-aging agent; a pigment; an anti-static agent; an anti-frizz agent; an antidandruff agent; a slip agent; an opacifier; a pearlizing agent and a salt. More preferably, the clear hair styling formulation of the present invention, optionally, further comprises at least one additional ingredient selected from the group consisting of an antimicrobial agent/preservative; a pH adjusting agent; a fragrance; a chelating agent; and salt.

Preferably, the clear hair styling formulation of the present invention optionally further comprises 0 to 5 wt % (more preferably, 0.05 to 4 wt %; still more preferably, 0.1 to 3 wt %; most preferably, 0.5 to 2 wt %), based on weight of the clear hair styling formulation, of an antimicrobial agent/preservative. More preferably, the clear hair styling formulation of the present invention optionally further comprises 0 to 5 wt % (more preferably, 0.05 to 4 wt %; still more preferably, 0.1 to 3 wt %; most preferably, 0.5 to 2 wt %), based on weight of the clear hair styling formulation, of an antimicrobial agent/preservative; wherein the antimicrobial/preservative is selected from the group consisting of phenoxyethanol, ethylhexyl glycerin, benzoic acid, benzyl alcohol, sodium benzoate, DMDM hydantoin, 2-ethylhexyl glyceryl ether, isothiazolinone (e.g., methylchloroisothiazolinone, methylisothiazolinone) and mixtures thereof. Most preferably, the clear hair styling formulation of the present invention optionally further comprises 0 to 5 wt % (more preferably, 0.05 to 4 wt %; still more preferably, 0.1 to 3 wt %; most preferably, 0.5 to 2 wt %), based on weight of the clear hair styling formulation, of an antimicrobial agent/preservative; wherein the antimicrobial/preservative is a mixture selected from the group consisting of (a) phenoxyethanol and ethylhexyl glycerin and (b) phenoxyethanol and an isothiazolinone (more preferably, wherein the antimicrobial/preservative is a mixture selected from the group consisting of (a) phenoxyethanol and ethylhexyl glycerin and (b) phenoxyethanol and methylisothiazolinone; most preferably, wherein the antimicrobial/preservative is a mixture of phenoxyethanol and ethylhexyl glycerin).

Preferably, the clear hair styling formulation of the present invention optionally further comprises 0 to 5 wt % (preferably, 0.1 to 4 wt %; more preferably, 0.5 to 3 wt %; most preferably, 0.75 to 2 wt %), based on weight of the clear hair styling formulation, of a pH adjusting agent. More preferably, the clear hair styling formulation of the present invention, optionally further comprises 0 to 5 wt % (preferably, 0.1 to 4 wt %; more preferably, 0.5 to 3 wt %; most preferably, 0.75 to 2 wt %), based on weight of the clear hair styling formulation, of a pH adjusting agent, wherein the clear hair styling formulation has a pH of 4 to 9 (preferably, 4.25 to 8; more preferably, 5 to 8; most preferably, 6.5 to 8).

Preferably, the pH adjusting agent is selected from the group consisting of at least one of citric acid, lactic acid, hydrochloric acid, aminoethyl propanediol, triethanolamine, monoethanolamine, sodium hydroxide, potassium hydroxide, amino-2-methyl-1-propanol. More preferably, the pH adjusting agent is selected from the group consisting of at least one of citric acid, lactic acid, sodium hydroxide, potassium hydroxide, triethanolamine, amino-2-methyl-1-propanol. Still more preferably, the pH adjusting agent includes triethanolamine. Most preferably, the pH adjusting agent is triethanolamine.

Preferably, the clear hair styling formulation of the present invention contains <0.1% (preferably, <0.01%; more preferably, <0.001%; most preferably, <detectable limit), based on weight of the clear hair styling formulation, of copolymers including structural units of maltodextrin and structural units of n-vinyl pyrrolidone.

Preferably, the clear hair styling formulation of the present invention contains <0.1% (preferably, <0.01%; more preferably, <0.001%; most preferably, <detectable limit), based on weight of the clear hair styling formulation, of copolymers including structural units of fructan polysaccharide.

Preferably, the clear hair styling formulation of the present invention contains <0.1% (preferably, <0.01%; more preferably, <0.001%; most preferably, <detectable limit), based on weight of the clear hair styling formulation, of copolymers including structural units of maltodextrin and structural units of dextrin.

Preferably, the clear hair styling formulation of the present invention contains <0.1% (preferably, <0.01%; more preferably, <0.001%; most preferably, <detectable limit), based on weight of the clear hair styling formulation, of copolymers including structural units of maltodextrin and structural units of maltodextrin.

Preferably, the method of styling hair of the present invention comprises: selecting a clear hair styling formulation of the present invention, applying the clear hair styling formulation to hair (preferably, mammalian hair; most preferably, human hair), and imparting a style to the hair (e.g., putting the hair in rollers, rolling the hair with an iron, flattening the hair with an iron). More preferably, the method of styling hair of the present invention, comprises: selecting a clear hair styling formulation of the present invention; wetting the hair (preferably, mammalian hair; most preferably, human hair) with water; applying the selected clear hair styling formulation to the wetted hair; and imparting a style to the hair (e.g., putting the hair in rollers, rolling the hair with an iron, flattening the hair with an iron). Most preferably, the method of styling hair of the present invention, comprises: selecting a clear hair styling formulation of the present invention; providing a shampoo; applying the shampoo to the hair (preferably, mammalian hair; most preferably, human hair); working the shampoo into the hair; rinsing the shampoo from the hair with water to provide clean hair; applying the selected clear hair styling formulation to the clean hair; and imparting a style to the hair (e.g., putting the hair in rollers, rolling the hair with an iron, flattening the hair with an iron).

Preferably, the method of styling hair of the present invention imparts a style to hair having a high humidity curl retention. More preferably, the method of styling hair of the present invention imparts a style to hair having a high humidity curl retention of greater than 50% (preferably, greater than 75%; more preferably, greater than 80%; most preferably, >90%) after eight hours at 25° C. in a 90% relative humidity environment.

Some embodiments of the present invention will now be described in detail in the following Examples.

### Synthesis S1-S6: Extruded Hydrolyzed Starch

The base starch was processed through a reactive extrusion process with high shear forces. The base starches processed through a reactive extrusion process with acid hydrolysis to provide hydrolyzed starches with the molecular weight and polydispersity noted in TABLE 2.

The Molecular weight analysis was done by GPC as follows.

Sample preparation: The starch samples were first dissolved in DMSO at 50 mg/ml and heated at 70° C. for an hour or until the solution became clear. The DMSO starch solutions were then further diluted to a concentration of 0.5-5 mg/mL with 20 mM NaCl solution at pH 11. The final test solutions were filtered through 0.45 μm syringe top PVDF filter before GPC analysis.

Instrumentation: A Waters 2690 system consisting of a pump, degasser, autosampler, column oven, and 2414 RI detectors operated at 40° C. System control, data acquisition, and data process were performed using Empower® 3 software (Waters, Milford, MA).

The GPC method used as noted in TABLE 1:

TABLE 1

| Column Set: | Tosoh Bioscience TSKgel GMPWxl and G3000PWxl | |
|---|---|---|
| | columns (7.8 mm ID × 30 cm) packed with 13 μm and 7 μm | |
| | particles, respectively | |
| Mobile Phase: | 20 mM NaCl, pH is adjusted to 11 with NaOH solution | |
| Flow Rate: | 1 | mL/min |
| Injection Volume: | 100 | μL |
| Column temperature: | 40° | C. |

| | |
|---|---|
| Column calibration: | Pullulan molecular weight standards from Agilent |
| | technology with molecular weight range from 708,000 |
| | Daltons to 180 Daltons |
| Detection: | Refractive Index (RI) at 40° C. |

TABLE 2

| | Base | | | |
|---|---|---|---|---|
| Synthesis | Starch | Mₙ (kDa) | M_{w} (kDa) | PDI |
| Unprocessed | Potato | >100 | >1,000 | — |
| S1 | Potato | 5.9 | 32 | 5.42 |
| S2 | Potato | 9.2 | 44 | 4.78 |
| S3 | Potato | 5.9 | 19 | 3.22 |
| S4 | Potato | 6.4 | 18 | 2.81 |
| S5 | Potato | 4 | 13 | 3.25 |
| S6 | Corn | 1.4 | 9.8 | 7.00 |

### Clarity Aqueous Starch Solutions

Aqueous solutions (3 wt %) of the ingredients noted in TABLE 3 were prepared in deionized water. The turbidity of each of the resulting aqueous solutions was measured with a turbidity meter. The meter was calibrated according to instructions included with the machine. A portion of the formulated product was slowly transferred to a clean glass vial. The glass vials were then centrifuged for 10 minutes at 2.5 rpm to remove any air bubbles. The samples were placed in the measurement column of the turbidity meter and after stabilization the turbidity of the sample was measured as recorded as noted in TABLE 3.

TABLE 3

| Ingredient | Turbidity (NTU) |
|---|---|
| Polyvinylpyrrolidone¹ | 1.89 |
| Maltodextrin/VP² | 1.08 |
| Hydrolyzed corn starch³ | >100 |
| Unprocessed potato starch | >100 |
| Synthesis S1 | 1.75 |
| Synthesis S2 | 1.62 |
| Synthesis S3 | 1.09 |
| Synthesis S4 | 1.16 |
| Synthesis S5 | 1.10 |
| Synthesis S6 | 0.52 |

¹PVP K90 from Ashland
²BIOSTYLE CGP polymer from Nouryon
³MAIZECARE ™ Style polymer from The Dow Chemical Company

Clarity Aqueous Blend of Starch with Carbomer

Aqueous blends containing the ingredient noted in TABLE 4 (3 wt %) and carbomer (1 wt %) (CARBOPOL 980 from Lubrizol) were prepared in deionized water. The turbidity of the resulting aqueous blends were measured with a turbidity meter. The meter was calibrated according to instructions included with the machine. The meter was calibrated according to instructions included with the machine. A portion of the formulated product was slowly transferred to a clean glass vial. The glass vials were then centrifuged for 10 minutes at 2.5 rpm to remove any air bubbles. The samples were placed in the measurement column of the turbidity meter and after stabilization the turbidity of the sample was measured as recorded as noted in TABLE 4.

TABLE 4

| Ingredient | Turbidity (NTU) |
|---|---|
| Polyvinylpyrrolidone¹ | 8.7 |
| Maltodextrin/VP² | 14.5 |
| Hydrolyzed corn starch³ | >100 |
| Unprocessed potato starch | >100 |
| Synthesis S1 | 69 |
| Synthesis S2 | 105 |
| Synthesis S3 | 18.9 |
| Synthesis S4 | 17.2 |
| Synthesis S5 | 5.6 |
| Synthesis S6 | 2.8 |

¹PVP K90 from Ashland
²BIOSTYLE CGP polymer from Nouryon
³MAIZECARE ™ Style polymer from The Dow Chemical Company

### Comparative Examples C₁-C4 and Examples 1-7: Hair Gel Formulations

Hair gel formulations according to Comparative Examples C₁-C4 and Examples 1-7 were prepared having the components noted in TABLE 5. In each of Comparative Examples C1-C4 and Examples 1-7, deionized water (50 g) was weighed into a 400 mL beaker outfitted with a stir bar. The beaker was placed on a magnetic stirrer and set to stir at 500 rpm. The carbomer (Carbopol 980 from Lubrizol) (1 g) was then added to the contents of the beaker with continued stirring until dispersed, at which point the stirrer setting was reduced to 200 rpm to remove the air bubbles.

Deionized water (39.9 g) was weighed into a 150 mL flask outfitted with a stir bar. The flask was then placed on a magnetic stirrer and set to stir. Disodium ethylenediaminetetraacetic acid (0.1 g) was added to the flask contents. Once the disodium ethylenediaminetetraacetic acid dissolved, glycerin (3 g) was added to the flask contents. Then the hair fixative polymer (5 g) as noted in TABLE 5 was added to the flask contents. Once the solution in the flask was clear, the preservative (Euxyl PE 9010 from Ashland) (1 g) was added to the flask contents.

The contents of the flask were slowly added to the contents of the beaker over a period of two minutes with continued stirring and the beaker contents were pH adjusted to 7-7.5 by addition of triethanolamine to provide the product hair gel formulations.

TABLE 5

| | Example (g) | | | | | | | | | |
|---|---|---|---|---|---|---|---|---|---|---|
| Ingredient | C1 | C2 | C3 | C4 | 1 | 2 | 3 | 4 | 5 | 6 |
| Deionized water | 89.9 | 89.9 | 89.9 | 89.9 | 89.9 | 89.9 | 89.9 | 89.9 | 89.9 | 89.9 |
| Carbomer¹ | 1.0 | 1.0 | 1.0 | 1.0 | 1.0 | 1.0 | 1.0 | 1.0 | 1.0 | 1.0 |
| Disodium EDTA | 0.1 | 0.1 | 0.1 | 0.1 | 0.1 | 0.1 | 0.1 | 0.1 | 0.1 | 0.1 |
| Glycerin | 3.0 | 3.0 | 3.0 | 3.0 | 3.0 | 3.0 | 3.0 | 3.0 | 3.0 | 3.0 |
| Preservative² | 1.0 | 1.0 | 1.0 | 1.0 | 1.0 | 1.0 | 1.0 | 1.0 | 1.0 | 1.0 |
| Polyvinylpyrrolidone³ | 5 | 0 | 0 | 0 | 0 | 0 | 0 | 0 | 0 | 0 |
| Maltodextrin/VP⁴ | 0 | 5 | 0 | 0 | 0 | 0 | 0 | 0 | 0 | 0 |
| Hydrolyzed corn starch⁵ | 0 | 0 | 5 | 0 | 0 | 0 | 0 | 0 | 0 | 0 |
| Unprocessed potato starch | 0 | 0 | 0 | 5 | 0 | 0 | 0 | 0 | 0 | 0 |
| Synthesis S1 | 0 | 0 | 0 | 0 | 5 | 0 | 0 | 0 | 0 | 0 |
| Synthesis S2 | 0 | 0 | 0 | 0 | 0 | 5 | 0 | 0 | 0 | 0 |
| Synthesis S3 | 0 | 0 | 0 | 0 | 0 | 0 | 5 | 0 | 0 | 0 |
| Synthesis S4 | 0 | 0 | 0 | 0 | 0 | 0 | 0 | 5 | 0 | 0 |
| Synthesis S5 | 0 | 0 | 0 | 0 | 0 | 0 | 0 | 0 | 5 | 0 |
| Synthesis S6 | 0 | 0 | 0 | 0 | 0 | 0 | 0 | 0 | 0 | 5 |

| | |
|---|---|
| Tetraethanol amine | final pH adjusted to 7-7.5 |

¹CARBOPOL 980 from Lubrizol
²EUXYL PE 9010 from Ashland
³PVP K90 from Ashland
⁴BIOSTYLE CGP polymer from Nouryon
⁵MAIZECARE ™ Style polymer from The Dow Chemical Company

### High Humidity Curl Retention

Hair tresses (medium to dark brown virgin hair, obtained from International Hair Importers) with an average length of 8 inches (prior to curling) and weighing 3.5 g were used for the curl retention experiments. The tresses were washed using a 9% sodium lauryl sulfate SLS solution, allowed to dry in a controlled temperature room overnight. The tresses were then treated with a hair gel formulation according to one of Comparative Examples C₁-C4 and Examples 1-7 (0.35 g) and then curled wet onto a 22 mm×70 mm curler and held in place with a bobby pin. After drying, the curlers were gently removed from the tresses and the curls were suspended by clips in humidity chamber at 25° C. and 90% relative humidity. The initial curl length was recorded. The lengths of the curled tresses were then recorded after 8 hours. Curl retention was defined as [(L(0)−L(t))/(L(0)−L(i))]×100%, wherein L(0) is the fully extended tress length, L(i) is the initial curl length and L(t) is the curl length after eight hours. Higher values for curl retention correspond to better humidity resistance performance. The observed curl retentions for the hair gel formulations are reported in TABLE 6.

TABLE 6

| | Curl retention |
|---|---|
| Hair gel formulation | after 8 hrs (%) |
| Comparative Example C1 | 10 |
| Comparative Example C2 | 20 |
| Comparative Example C3 | 80 |
| Comparative Example C4 | — |
| Example 1 | 86 |
| Example 2 | 90 |
| Example 3 | 70 |
| Example 4 | 86 |
| Example 5 | 93 |
| Example 6 | 95 |

We claim:

1. A clear hair styling formulation, comprising:
a dermatologically acceptable vehicle; and
a hair fixative polymer; wherein the hair fixative polymer is a hydrolyzed starch;
wherein the hydrolyzed starch has a weight average molecular weight of 5,000 to 50,000 Daltons.

2. The clear hair styling formulation of claim 1, further comprising a thickener.

3. The clear hair styling formulation of claim 2, further comprising a plasticizer.

4. The clear hair styling formulation of claim 3, wherein the clear hair styling formulation has a turbidity, when measured by nephelometry at a concentration of 1 wt % hair fixative polymer and a pH of 7 to 7.5, of 0 to 20 Nephelometric Turbidity Units (NTU).

5. The clear hair styling formulation of claim 4, wherein the thickener is selected from the group consisting of a crosslinked poly(acrylic acid) polymer, a hydrophobically modified crosslinked poly(acrylic acid) polymer and a mixture thereof.

6. The clear hair styling formulation of claim 5, wherein the thickener is a crosslinked poly(acrylic acid) polymer.

7. The clear hair styling formulation of claim 6, wherein the weight ratio of thickener to hair fixative polymer is 1:5 to 1:25.

8. The clear hair styling formulation of claim 7, further comprising an additive selected from the group consisting of an antimicrobial agent/preservative; a soap; a colorant; pH adjusting agent; an antioxidant; a humectant; a wax; a foaming agent; an emulsifying agent; a colorant; a fragrance; a chelating agent; a bleaching agent; a lubricating agent; a sensory modifier; a vitamin; a bioactive agent; a pigment; an anti-static agent; an anti-frizz agent; an antidandruff agent; a slip agent; an opacifier; a pearlizing agent and a salt.

9. The clear hair styling formulation of claim 8, wherein the clear hair styling formulation is selected from the group consisting of a hair spray, a hair fixative, a hair mousse, a hair gel/cream/paste, a hair volumizing composition, a hair leave-on cream, a hair mask, a hair lotion, a hair serum, a hair oil, a hair clay, a hair pomade, a hair wax, a hair powder and a dry shampoo

10. A method of styling hair, comprising:
providing a clear hair styling formulation of claim 1;
applying the clear hair styling formulation to hair; and
imparting a style to the hair.
